(19) United States
(12) Patent Application Publication
(10) Pub. No.: US 2026/0248140 A1
(43) Pub. Date: Aug. 27, 2026

(54) BIOFILM DEGRADING COMPOSITION

(71) Applicant: ONELIFE, Louvain-la-Neuve (BE)

(72) Inventor: Martine Weickmans, Héron (BE)

(73) Assignee: ONELIFE S.A., Louvain-la-Neuve (BE)

(21) Appl. No.: 18/877,375

(22) PCT Filed: Sep. 25, 2023
(86) PCT No.: PCT/EP2023/076377

§ 371 Date: Dec. 20, 2024

(30) Foreign Application Priority Data
Sep. 23, 2022 (BE) 2022/5764

(51) Int. Cl. A01N 63/50; A01N 25/02; A01N 25/30; A01P 1/00

(52) U.S. Cl. CPC A01N 63/50; A01N 25/02; A01N 25/30; A01P 1/00

(57) ABSTRACT

A biofilm degrading composition comprising a DNAse, uses thereof, and a method of degrading biofilms comprising the application of this composition.

### TECHNICAL FIELD

The present invention relates to a composition for biofilm degradation comprising a specific DNAse.

### PRIOR ART

In many fields of activity, such as the agrifood sectors, the communities, the medical and veterinary sectors, the cosmetics industries, pharma, life science or biotechnology sectors, problematic contamination due to the presence of microorganisms is observed very frequently.

These biofilms can be treated in different ways, but it remains difficult to ensure that the treatment that is to be applied will be effective. In particular, biocidal molecules or antibiotics, which are known to destroy bacteria in planktonic form, are very often ineffective against these same bacteria in the form of a biofilm.

Furthermore, in practice, and unlike in the laboratory, the composition of a biofilm contaminating a surface is very generally unknown.

Finally, applications in the agrifood or medical field, or even for GMP (Good Manufacturing Practice) production, impose constraints as to the compounds that can be used, which complicates the treatment of biofilms. For example, pipes are difficult to disinfect due to reduced access. Likewise, medical instruments, such as endoscopes, are difficult to disinfect, due to (i) their surface, which exhibits many irregularities, and (ii) the impossibility of applying to them many of the usual physical or chemical treatments, such as heat treatments, products which would damage them or would be incompatible with the subsequent use in a patient.

Different types of enzymes have been tested and used successfully for the degradation of biofilms. However, some biofilms are not suitably destructured with a single enzyme activity, whereas mixtures of enzymes, especially when proteases are present, do not necessarily allow liquid compositions that are stable over time. In addition, as will be described below, for a biofilm of given composition, the inventors have noted that the current enzymatic compositions too often exhibit significant variations in terms of effectiveness, which complicates the work to arrive at an optimal formulation.

Patent application WO2022079318 describes the use of a particular nuclease (Denarase), with increased effectiveness.

Patent U.S. Pat. No. 10,954,497 describes a category of DNAses HNH having a GYS motif and their effectiveness when formulated for the destruction of biofilms.

### BRIEF SUMMARY OF THE INVENTION

The present invention relates to a liquid composition for the prevention or removal of a biofilm, comprising one or more surfactant(s), one or more sequestering agent(s) and a phosphodiesterase having deoxyribonuclease (DNAse) activity, said DNAse being one or more DNAse(s) of HNH type and/or having at least 95% identity with one of the sequences SEQ ID NO: 1 to SEQ ID NO:13 (or SEQ ID NO: 1 to SEQ ID NO:18).

The present invention further relates to the use of this composition for the removal of a biofilm comprising a lactic acid bacterium selected from Lacobacillus sp, Pediococcus sp, Lactococcus sp. Streptococcus sp, Teragenococcus sp, Leuconostoc sp, Oenococcus sp, Bifidobacterium sp,
and/or Listeria monocytogenes, Stenotrophomonas maltophilia, Bacillus cereus, Bacillus subtilis,
and/or Pseudomonas fluorescens, Pseudomonas aeruginosa, Staphylococcus aureus, Escherichia coli, Klebsiella pneumoniae, Enterobacter cloacae, Citrobacter freundii, Staphylococcus epidermidis or Clostridium sp.

The present invention also relates to this liquid composition (for pharmaceutical use) for the removal of a biofilm present on a tissue of a patient, said tissue preferably being the oral cavity, a mucous membrane, a wound or in contact with an implant.

The present invention also relates to a method for the removal of a biofilm on a surface comprising applying this liquid composition to said surface.

### DETAILED DESCRIPTION OF AN EMBODIMENT OF THE INVENTION

The inventors sought to improve the compositions against biofilms, in particular to obtain compositions whose spectrum of activity is sufficiently broad and constant, and which ensures a considerable reduction in the number of microorganisms protected by the biofilm.

To do this, the inventors identified a DNAse which is particularly effective against certain types of biofilms.

In addition, depending on the biofilms, this DNAse is highly effective, either alone or in synergy with other enzymatic activities.

Moreover, the inventors succeeded in formulating this DNAse so that it retains its activity, including over time (even in the presence of a protease in the composition or of sequestering agents), which first of all allows reasonable commercial logistics between the time when the liquid (aqueous) composition is formulated and the time when it will be used.

The composition according to the invention is preferably used on surfaces. In the context of the present invention, the term “surface” is preferably understood in the broad sense, therefore including the internal surface of a pipe, or else surfaces of reusable medical devices, in particular endoscopes and surgical instruments.

Indeed, the inventors tested the effect of this DNAse alone, compared with other nucleases known for their remarkable activity, such as Denarase (as in WO2022079318), and the effect of this DNAse in synergy with other enzymes, either in a mixture or in sequential application, and noted an increased or more constant effectiveness, depending on the type of biofilm to be treated. Indeed, to their surprise, the inventors compared, under well-controlled laboratory conditions, the effect on biofilms of the compositions (protease+amylase+lipase+detergent+mild sequestering agent) without this DNAse and of the same compositions with the DNAse of the invention, and, in the case of the composition without this DNAse, they sometimes noted good results and sometimes poor results, whereas the compositions enriched with the DNAse of the present invention always showed very good results. Thus, it took many tests, and repeated many times, to clearly identify the effect of the DNAse of the present invention, and the results shown below are among the most spectacular.

### SEQUENCE LISTING

Sequences 1 to 11 and 14 to 18 reproduce DNAses according to the invention. These are DNAses of the HNH type.

Sequence 12 is the consensus sequence in its broadest understanding.

Sequence 13 is a consensus sequence incorporating preferred amino acids at key positions, especially in the central part of the enzyme.

In sequences 12 and 13, the amino acids that may be subject to variation are mentioned under the letter “X” and their structure is repeated below. Certain positions are preferentially occupied by specific amino acids, which is also listed below:

SEQ ID NO: 12
XPXXXPSKXXXQXQLXXLXVXXEXXMXGYSRXXFPHWXXQGXGCXTRQXV
LXRDADXXSGXCPXTXGXWYSYXDGXXXXXPSXXDXDHXVPLAEAWRSGA
SSWXTXXRXXFANDLXGXQLIAVXASXNRXKGDQDPSTWXPXRXXAXCXY
XKXWXXTKXXXXLXLQSXEKXXLXXMLXXCXY
SEQ ID NO: 13
LPPGTPSKSXAQSQLNALXVXXEXSMTGYSRXXFPHWSSQGGGCDTRQVV
LKRDADXXSGXCPVTSGKWYSYFDGXXFTNPSDLDIDHIVPLAEAWRSGA
SSWTTSKRXXFANDLNGPQLIAVXASXNRXKGDQDPSTWQPPRAAARCAY
SKWWISTKYKWGLSLQSXEKXXLXXMLXXCAY

1: L, F, I, T, P; preferably L or I, preferably L;
3: P, S; preferably P;
4: G, E, V, preferably G, V, preferably G;
5: T, I; preferably T;
9 S, A, preferably S;
10. (sequences 12 and 13). A, E, Q, T, preferably E, Q;
11. A, S; preferably A;
13. S, T; preferably S;
16. N, D; preferably N;
17 A, S, G; preferably A, S;
19 (sequences 12 and 13). T, A; preferably T;
21 (sequences 12 and 13). K, Q; preferably K;
22 (sequences 12 and 13). A, P, T, S; preferably P or S;
24 (sequences 12 and 13). D, S, G; preferably D;
25. P, T, A, S; preferably P, S;
27. T, S; preferably T;
32. (sequences 12 and 13). D, N;
33. (sequences 12 and 13). L, H, K;
38. N, S, I, T;
39. S, G, preferably S;
42. S, G, N;
45. D, N;
49. I, L, V, M;
52. Q, K;
57 (sequences 12 and 13), Y, S; preferably Y;
58 (sequences 12 and 13). Y, F, preferably Y at least one residue Y at positions 57/58, preferably positions 57 and 58 are occupied by 2 residues Y;
59. S, T; preferably S;
61 (sequences 12 and 13). N, A, T, S, D; preferably A;
64. V, T; preferably V;
66. S, T; preferably S;
68. R, K, S; preferably R, K;
73. F, Y;
76 (sequences 12 and 13). I, V;
77 (sequences 12 and 13). V, I, T, K;
78. V, F;
79. T, Y;
80. S, D, N; preferably S;
83. E, D, N; preferably E, D;
84. I, L;
86. I, V; preferably I
89. I, V; preferably I; at least 1 I at positions 86/89;
104. S, T; preferably T;
106. E, S, Q, A, T;
107. K, Q; preferably K;
109. (sequences 12 and 13). E, K, Q, R; preferably Q or R, preferably R
110. (sequences 12 and 13). E, S, A, N, D; preferably N or D, preferably N
116. N, T, G, S; preferably N, S;
118. P, S, preferably P;
124 (sequences 12 and 13). S, T;
127 (sequences 12 and 13). T, S, V; preferably S, V;
130 (sequences 12 and 13). S, A; preferably S;
140. Q, K; preferably Q;
142. P, T; preferably P;
144. A, V, S, Y;
145. A, G, S; preferably G;
147. R, H, K, A; preferably R;
149. G, A;
151. S, A; preferably A;
153. W, M; preferably W;
155. I, V. preferably I;
156. N, S; preferably N;
159. H, Y, S; preferably Y, H;
160. R, V, K; preferably R, K, preferably R;
161. W, Y; preferably W;
162. D, G, N, preferably G, D;
164. S, H, N;
168 (sequences 12 and 13). S, A; preferably S;
171 (sequences 12 and 13). S, T, N; preferably S;
172 (sequences 12 and 13). S, A, G, preferably S, A;
174 (sequences 12 and 13). Q, E; preferably Q;
175 (sequences 12 and 13). T, S, G;
178 (sequences 12 and 13). N, D; preferably N;
179 (sequences 12 and 13). T, G, S; preferably T, S, preferably T;
181. S, A.

Thus, a first subject of the present invention relates to a liquid composition for the prevention or removal of a biofilm, comprising one or more surfactant(s), one or more sequestering agents and a phosphodiesterase having a deoxyribonuclease (DNAse) activity, said DNAse being one or more DNAse(s) of the HNH type (having a GYS unit) and/or having at least 95% identity with one of the sequences 1 to 13, or even with the DNAses of sequences 1 to 18.

Preferably, said liquid composition comprises at least 7% by weight of water, preferably at least 10% of water, or else at least 20% of water. Preferably, in this composition, the DNAse(s) has (have) at least 95% identity with one of the sequences 1 to 13, or even with the DNAses of sequences 1 to 18.

In the context of the present invention, the identity is preferably measured by an alignment of a test sequence with one of sequences 1 to 13 (or 1 to 18) over their entire length (182 amino acids). The default BLASTp parameters are advantageously used, such as a Blosum62 matrix, a size 6 “word”, a gap penalty of 11 and a gap extension penalty of 1. The percentage of identity is thus unambiguously calculated. Advantageously, in the above calculation, a “gap” will be counted as a single difference, regardless of its length.

Similarly, when a DNAse is to be compared with sequences 12 and 13, a global alignment is carried out as described above, and an amino acid of the sequence to be compared which would be identical to the variants of sequences 12 or 13 listed at the different positions marked with the symbol “X” is counted as identical. For example, a sequence to be compared would be aligned with the sequence 12 or 13. If the amino acid of this sequence to be compared to the position corresponding to position 10 of sequence 12 or 13 is an alanine, it will be considered identical.

A sequence which would have an equivalent enzymatic activity, but which would be longer (e.g. addition of sequences for purification or of sequences for increasing stability, or even of signal sequence), or shorter, is also covered by the present invention, provided that the enzyme in question includes at least 95% of one of sequences 1 to 13 (or 1 to 18).

Alternatively, the identity can be deduced after a global alignment as above and by applying a tolerance of less than 10, less than 9, less than 8, less than 7, less than 6, less than 5, less than 4, less than 3 variations or “gaps” between a target sequence and any one of sequences 1 to 13 (or 1 to 18).

### Additional Enzymes

Advantageously, the composition further comprises one or more enzymatic activities chosen from one (or more) proteases, a laccase, an amylase, a lipase, a cellulase, a mannanase, a β-1,6-N-acetylhexosaminidase activity (Dispersin B) and a mixture thereof, preferably a protease (or even several proteases) and/or β-1,6-N-acetylhexosaminidase (Dispersin B), preferably a protease (or even several proteases) and/or an β-1,6-N-acetylhexosaminidase (Dispersin B) and another enzyme, or several other enzymes, chosen from a laccase, an amylase, a lipase, a cellulase and a mannanase.

For example, a preferred composition comprises the DNAse described above and one (or more) proteases, one (or more) amylases and one (or more) cellulases.

A preferred composition may also comprise the DNAse described above and another nuclease, as well as, advantageously, a protease (or even a cellulase and/or an amylase and/or a second protease).

One or more enzymes catalyzing oxidation-reduction reactions may advantageously be added.

Indeed, even if the addition of several enzymatic activities presents difficulties, in particular as regards stability, for example because of the sequestering agents (see below) or the endogenous activity of the protease, the inventors noted that the presence of several different enzymatic activities offers a broader spectrum of activity, which is advantageous since, in general, the composition of the biofilms to be treated is unknown, or even the biofilms to be treated comprise several different structural elements: the effect, already remarkable, of the DNAse of the invention is further reinforced by the additional enzymatic activities.

### Other Components

The surfactant(s) is (are) not particularly limited. Anionic, neutral and/or zwitterionic surfactants may be present. The inventors have noted that surfactants of alkyl polyglucoside type work well, as do zwitterionic surfactants of alkyl aminoxide type (e.g. with 12 and/or 14 and/or 16 and/or 18 carbons).

When the composition is to be applied to vertical surfaces, for example a siphon, a foaming surfactant is advantageously incorporated. In addition, an agent reinforcing the foaming character, such as betaine derivatives, and/or zwitterionic surfactants having a quaternary amine are advantageously added.

Typically, these surfactants are present in a (final) content of at least 1%, by weight:volume. Advantageously, their content does not exceed 15% (by weight:volume; sum of the weights of the surfactants:volume).

Preferably, in the composition according to the invention, the sequestering agent(s) is (are) selected from the group consisting of citrate, carboxymethylinulin, a phosphate, a phosphonate, and amino acid derivatives (tetrasodium glutamate and diacetate, GLDA; trisodium methylglycine diacetate, MGDA), sodium iminodisuccinate; IDS, gluconate (e.g. sodium gluconate) and mixtures thereof. Preferably, the sequestering agent(s) is (are) selected from the group consisting of citrate, carboxymethylinulin, a phosphate, a phosphonate, gluconate and mixtures thereof. Preferably, they are gentle sequestering agents. The inventors noted that these sequestering agents do not interfere here with the activity of the DNAse of the invention, nor with that of the other enzymes listed above (when they are present).

Advantageously, the composition according to the invention also comprises glycerol and/or monopropylene glycol. The inventors noted that this type of molecule increases the stability of the composition according to the invention. An advantageous content is approximately 5 to 30% by weight of glycerol and/or monopropylene glycol:volume of the solution, preferably 7 to 15% (weight:volume), such as 8 to 12% (weight:volume), or approximately 10%±0.5% (weight of glycerol and/or monopropylene glycol:volume). Sorbitol may also be incorporated for the same purpose. However, when the composition is intended for administration to a patient (implant, mucous membranes of the digestive or respiratory systems), it advantageously comprises no glycerol or monopropylene glycol, or in any case less than 5%, or even less than 1%, or else less than 0.1% (weight of glycerol and/or monopropylene glycol:volume of the composition).

Conversely, or in addition, the liquid composition according to the invention advantageously comprises (in addition to glycerol and/or monopropylene glycol) water, for example at least 5% by weight, preferably at least 10% by weight.

In addition, advantageously, the liquid composition according to the invention is present according to a concentrated formulation, which is diluted (e.g. 10 times, 100 times or more) in water just before use.

However, certain compositions are advantageously directly formulated “ready-to-use”; for example, compositions comprising foaming surfactants (e.g. as described above for the treatment of siphons); a more detailed description of this will be provided below. Foaming surfactants include those with an HLB (Hydrophilic-Lipophilic-Balance) value between 3 and 8. Alternatively, several anionic or zwitterionic surfactants may be used, preferably in synergy with a betaine derivative, such as 1-propanaminium, 3-amino-N-(carboxymethyl)-N,N-dimethyl-, N-acyl (the acyl being a hydrocarbon chain with an even number of carbons, for example 18),

Similarly, advantageously, the composition according to the invention also comprises a preservative, preferably an isothiazolinone such as benzisothiazolinone, or (2-)phenoxyethanol. Conversely, the compositions intended to be administered to a patient (implant, mucous membranes of the digestive or respiratory systems) advantageously do not comprise a preservative.

Advantageously, the composition according to the invention comprises at least 40% (by volume:volume) water. This is a composition that must not be diluted too much before use. On the other hand, an excessively high water content risks damaging the stability of the composition over time.

Another related aspect of the present invention relates to a “ready-to-use” composition (with at least one DNAse of the HNH type and/or having at least 95% identity over the entire sequence with any one of Sequences 1 to 13, or 1 to 18), comprising at least 90%, or even at least 95% water (weight of the water:weight of the composition) and from 1 to 10% (for example from 2 to 5%) of the other components (sum of the weights of the components other than water:total weight of the composition): the DNA according to the invention, the optional other enzymes (preferably one or more proteases; amylase, optionally a lipase, or even the other enzymes described above), a sequestering agent and one or more surfactants, or even one or more filler compound (carrier) if present.

Preferably, such a “ready-to-use” composition comprises a foaming surfactant and/or a betaine derivative as described above.

Another related aspect of the present invention relates to the use of the composition according to the invention (with at least one DNAse of the HNH type and/or having at least 95% identity over the entire sequence with any one of Sequences 1 to 13, or 1 to 18) for the removal of a biofilm comprising a lactic acid bacterium chosen from Lacobacillus sp, Pediococcus sp, Lactococcus sp, Streptococcus sp, Teragenococcus sp, Leuconostoc sp, Oenococcus sp, Bifidobacterium sp, and/or
Listeria sp (or L. monocytogenes), Stenotrophomonas maltophilia, Bacillus sp. (B. cereus, B. subtilis) and/or Pseudomonas sp. (e.g. Pseudomonas fluorescens or Pseudomonas aeruginosa),
Staphylococcus aureus, Escherichia coli, Klebsiella pneumoniae, Enterobacter cloacae, Citrobacter freundii, Staphylococcus epidermidis, Clostridium sp.

Indeed, in the case of biofilms comprising the microorganisms listed above, the inventors noted the superiority of the DNAse according to the invention compared with other nucleases, even nucleases considered excellent, this superiority being measured either alone or in synergy with other enzymatic activities.

Another related aspect of the present invention relates to a (pharmaceutical) composition comprising the DNAse (or several DNAses) according to the invention (with at least one DNAse of the HNH type and/or having at least 95% identity over the entire sequence with any one of Sequences 1 to 13, or 1 to 18) for the removal of a biofilm
present on a tissue of a patient, said tissue being preferably the oral cavity, a mucous membrane, a wound,
or in contact with an implant.

In the context of the present invention, the term “mucous membrane” preferably relates to a mucous membrane chosen from the mucous membranes of the respiratory system (nasal cavities, bronchi), of the urogenital system and of the digestive system.

Preferably, this biofilm present on the tissue of a patient comprises Pseudomonas aeruginosa, Staphylococcus aureus, Escherichia coli, Klebsiella pneumoniae, Enterobacter cloacae, Citrobacter freundii, and/or Staphylococcus epidermidis

Advantageously, this pharmaceutical composition is produced according to “GMP” standards, which means that the compounds are chosen so as not to cause harmful effects to the patient (e.g., especially for implants or the mucous membranes of the digestive or respiratory systems, as little glycerol or propylene glycol as possible; as little as possible of preservative or other toxic agent for the patient), and that the production steps follow a strict protocol validated by independent authorities (health authorities, hospital ethics committees, . . . ).

Another related aspect of the present invention relates to a method for the removal of a biofilm on a surface comprising the application on this surface of the liquid composition according to the invention, comprising the DNAse (or several DNAses) according to the invention (with at least one DNAse of the HNH type and/or having at least 95% identity over the entire sequence with any one of Sequences 1 to 13, or 1 to 18).

A variant of this method is particularly advantageous for the treatment of medical instruments, in which the medical instrument is treated with the liquid composition according to the present invention comprising the DNAse described above (often in synergy with one or more surfactants, a sequestering agent and other enzymatic activities described above, in particular a protease, but also an amylase, or even at least one fourth enzyme), then the instrument is rinsed, then disinfected using a biocidal molecule, for example peracetic acid. Then, if the medical instrument has a delicate composition, for example, an endoscope, it is simply dried. On the other hand, if the instrument can be subjected to a sterilizing heat treatment (e.g. surgical instruments), the latter is advantageously carried out in addition. A sterilizing heat treatment is preferably a treatment in an autoclave at a temperature above 100° C. and at a pressure above 1 bar, the atmosphere advantageously being saturated with water. Another advantageous sterilizing heat treatment is pasteurization.

A related aspect of the present invention relates to a composition for the prevention or treatment of biofilms present on a substantially vertical surface comprising:
one or more enzymatic activities chosen from a DNAse (e.g. that of WO2022/079315 and/or of classes EC 3.1.30 and 3.1.31, or the DNAse described above of the HNH type and/or having at least 95% identity over the entire sequence with any one of Sequences 1 to 13, or 1 to 18), a protease, a polysaccharidase (e.g. amylase, Dispersin B, cellulase, mannanase), a lipase, a laccase and mixtures thereof;
one or more surfactants comprising at least one foaming surfactant.

Preferably, this composition further comprises a surfactant having a quaternary amine, said surfactant preferably being zwitterionic and/or a betaine derivative, preferably of structure RN⁺(CH₃)₂CH₂COO⁻. The group R is preferably a linear alkyl or an alkylamidopropylbetaine, such as cocoamidopropylbetaine. Optionally, the carboxyl group is substituted by a sulfonate group, and/or a hydroxyl group is grafted onto the alkyl chain. The (linear) alkyl chain preferably has a size of between 2 and 18 carbon atoms, preferably between 12 and 16 carbon atoms.

Another related aspect of the present invention relates to a method for the prevention or treatment of a biofilm on a substantially vertical surface comprising the successive steps of
applying the composition for the prevention or treatment of biofilms present on a substantially vertical surface as described above to said substantially vertical surface for a predetermined time;
adding a microbiocidal agent for a predetermined time.

This method is particularly advantageous when the vertical surface is a siphon, preferably the emergent part of said siphon.

### EXAMPLES

Two different biofilms were tested, so as to encounter a variety of microorganisms: a biofilm made of Lactococcus lactis and a biofilm made of Pseudomonas fluorescens. These biofilms were generated by applying bacterial suspensions on agar at 37° C. in multiwell plates (24 wells) for 48 h (Pseudomonas) or 24 h (Lactococcus). The microorganisms are quantified by spectrometry, here at 590 nm (570 nm is also OK) following staining with “Crystal Violet”.

Different conditions are provided for each plate, which is shown diagrammatically in Table 1 below:

TABLE 1
Overview of the different test conditions.
Each condition is therefore in triplicate.

| Positive | Positive | Positive | Negative | Negative | Negative |
|---|---|---|---|---|---|
| control | control | control | control | control | control |
| Cip | Cip + | Cip + | A1 + 10 | A1 + 10 + | A1 + 10 + |
| | Denarase | DNAse | | Denarase | DNAse |
| Cip | Cip + | Cip + | A1 + 10 | A1 + 10 + | A1 + 10 + |
| | Denarase | DNAse | | Denarase | DNAse |
| Cip | Cip + | Cip + | A1 + 10 | A1 + 10 + | A1 + 10 + |
| | Denarase | DNAse | | Denarase | DNAse |

Bacteria are seeded in all the wells except for the “negative controls”.

All solutions are sterile and are added very gently so as not to cause mechanical disturbances to the biofilms.

The wells of the “positive control” and “negative control” conditions were incubated with 1 mL of physiological saline solution.

The other wells were incubated for 30 minutes at 45° C. with 1 mL of the following solutions (the percentages below are by weight:volume):
Biorem-CIP (Realco): 1%;
Denarase and DNAse: 0.01% enzyme solutions, to ensure normalized activity;
Biorem A1+10: the solutions sold by Realco, diluted to 0.25% (A1) and 0.05% (Biorem 10). These solutions comprise detergents/surfactants (>1% C6 alkyl glucoside), sequestering agents (a phosphonate; >15%), and several enzymatic activities, including a protease, an amylase, and a laccase.

Biorem CIP also contains detergents/surfactants (>1% C6 alkyl glucoside), sequestering agents (>1% sodium citrate), and a dispersant and several enzymatic activities, including a protease, an amylase and a laccase. The composition was applied at 1%.

Biorem compounds are also described, for example in patent application WO2012048758A1 and in the corresponding patents.

Then the wells are gently emptied, taking care not to remove the biofilms or their fragments. After rinsing with physiological water, the plate containing the biofilms is dried in an oven for one hour at 45° C. Once the biofilm has dried, the Crystal Violet (0.1%) is added for a 15-minute incubation at room temperature, followed by three rinsings with distilled water. Finally, the wells are filled with 44% acetic acid for a one-hour incubation at room temperature. The absorbance measurement at 590 nm can now be carried out. If the Crystal Violet concentrations are too high and the spectrophotometer saturates then a one in ten dilution is carried out.

The results for Lactococcus lactis are given in Table 2 below:

TABLE 2
Effectiveness of the different nucleases for the destruction
of biofilms consisting of Lactococcus lactis and
synergy with an enzymatic composition.

| | Cip + | Cip + | | A1 + 10 + | A1 + 10 + |
|---|---|---|---|---|---|
| CIP | Denarase | DNAse | A1 + 10 | Denarase | DNASE |
| 60.6% | 66.5% | 71.3% | −4.8% | 4.5% | 47.0% |
| 59.4% | 61.1% | 63.7% | −2.5% | 0.0% | 55.2% |
| 49.9% | 53.8% | 65.1% | 10.4% | 5.4% | 61.1% |

The inventors also tested, in another series of experiments, the effect of DNAse alone or of Denarase alone, and observed a very good effectiveness of DNAse alone on Lactococcus lactis, which is further increased by about 20% by Biorem, whereas Denarase alone has only a marginal activity.

The results for Pseudomonas fluorescens are given in Table 3 below:

TABLE 3
Effectiveness of the different nucleases for the destruction
of biofilms consisting of Pseudomonas fluorescens and
synergy with an enzymatic composition.

| | Cip + | Cip + | | A1 + 10 + | A1 + 10 + |
|---|---|---|---|---|---|
| CIP | Denarase | DNAse | A1 + 10 | Denarase | DNASE |
| 84.4% | 71.8% | 75.5% | 19.0% | 44.1% | 70.0% |
| 81.3% | 73.6% | 69.7% | 13.0% | 42.7% | 66.3% |
| 71.0% | 70.5% | 73.5% | 17.0% | 46.8% | 63.4% |

Pseudomonas fluorescens and synergy with an enzymatic composition.

As in the case of L. lactis, the inventors tested the effect of DNAse alone in a second series of experiments and observed a strong activity of DNAse alone, which is somewhat increased by Biorem.

From this, the inventors conclude that the DNAse of the invention is clearly superior, especially in synergy with the less intense conditions of Biorem A1+10, compared to Biorem CIP. Advantageously, for Lactococcus lactis, where the effectiveness of Biorem CIP is somewhat less pronounced, the synergy with DNAse is better demonstrated.

Another series of experiments was carried out in synergy with other enzymatic compositions (Enzimed Prevent, which comprises a protease, a cellulase, an amylase and a mannanase; the first 2 columns of the tables below, without or with the DNAse of the present invention), and a comparison was carried out with other commercial compositions: alkaline detergent compositions with a protease activity (3rd column; DAP), i.e. an enzymatic degreasing cocktail, two separate commercial formulations (4th and 5th column; DE1 and DE2), a multienzymatic disinfecting detergent containing a quaternary ammonium (6th column; DMQ) or a non-enzymatic disinfection composition for endoscopes (7th column; DME). The compositions were tested at a concentration of 0.5%.

The biofilm of the test species is formed in a 96-well plate. The enzymatic detergents are then diluted to their usual concentration in water of standard hardness (demineralized water) and incubated on the biofilm for 1 h at 37° C. The amount of biomass remaining after treatment and washing of the biofilm is then evaluated by staining with Crystal Violet (2% solution of sigma).

This test is carried out on reference strains in a 96-well plate, namely:
S. aureus ATCC33591: 24 h biofilm (TGN)
E. coli ATCC25922: 48 h biofilm (LB)
P. aeruginosa ATCC27853 and PAO1: 48 h biofilm (TGN)

### Solutions:

$$TGN ( TSB + 1 \% glucose + 2 \% NaCl )$$

LB (for E. coli)

“Distilled” water: sterilized by filtration (0.22 micron), ultimately containing 1.25 mM MgCl2, 2.5 mM CaCl2, 3.33 mM NaHCO₃.

2% Crystal Violet (Crystal Violet solution, Sigma-Aldrich, ref: HT90132-1L)

Acetic acid 66% (glacial acetic acid 100%, ref: K48283163635, Millipore)

Dishes of Tryptic soy agar (TSA; BD benelux-ref 254086)

The solutions were preheated to 37° C., so as to avoid thermal shocks to the biofilms.

### Method:

D0: Launch an “overnight” culture. 5 mL TGN+20 μL of bacteria. Incubate for 18 hours at 37° C. with slow stirring, at 130 rpm
D1: Preparation

Add 200 mL of the inoculum (OD 0.05) to each well of a 96-well plate following the plate plan (VWR).

Do a noncontamination test of the liquid preculture

Streak the preculture on a TSA dish. Incubate overnight @37° C.

The next day, ensure that there is no contamination.

### Biofilm Growth:

For S. aureus 24 h@37° C.

Incubate the plate at 37° C. for 24 h in a closed dish to avoid evaporation. Note the time to have a 24 h biofilm exactly.

For E. coli: 48 h with change of medium after 24 h@37° C.:
Incubate the plate at 37° C. for 24 h in a closed dish to avoid evaporation (LB not TGN).
Gently remove the medium by pipetting
Add 200 μL of preheated LB
Incubate for an additional 24 h@37° C. in a closed dish to avoid evaporation.

For P. aeruginosa: 48 h@37° C.
Incubate the plate at 37° C. for 48 h in a closed dish to avoid evaporation. Note the time to have a 48 h biofilm exactly.
D2: After biofilm growth
Empty the culture medium of the biofilm plates by suction, avoiding touching the biofilm
Rinse the plates 1× with preheated sterile PBS (remove PBS after 30 sec)
Enzyme treatment:
   Prepare the enzyme solutions at the desired concentrations according to the annexed preparation procedure
   Add 230 μL of the enzyme solutions in empty 96-well plates according to the plate plan
   Heat these plates in a water bath at 37° C. or in the oven for 15 min
   Transfer 200 μL of heated enzymes into the wells of the 96-well plates comprising the biofilms by pipetting horizontally
   Incubate the plates at 37° C. for 1 h
   Empty enzymes from treated plates by suction
   Rinse 2× plates with preheated sterile PBS, between each step, remove PBS by suction Staining the biomass with Crystal Violet:
   Dry the plates overnight at 60° C. to fix the biofilm
   Add 200 mL of 2% Crystal Violet solution to the plates (including negative control)
   Incubate the plates at room temperature for 15 min
   Empty the plates and rinse them at least 5× with distilled water
   Drain the plates for a few minutes to remove rinsing water from the wells
   Add 200 μL of 66% acetic acid to the plates (including negative control)
   Incubate the plates at room temperature for at least 1 hour
   Measure the absorbance values at spectramax (570 nm) and carry out dilutions in acetic acid if certain plate values are >2

| | | | | | | |
|---|---|---|---|---|---|---|
| Prevent | Prevent | DAP | DE1 | DE2 | DMQ | DME |
| | DNase | | | | | |
| Prevent | Prevent | DAP | DE1 | DE2 | DMQ | DME |
| | DNase | | | | | |
| Prevent | Prevent | DAP | DE1 | DE2 | DMQ | DME |
| | DNase | | | | | |
| Prevent | Prevent | DAP | DE1 | DE2 | DMQ | DME |
| | DNase | | | | | |
| Average | Average | Average | Average | Average | Average | Average |

### Experiment 1

S. a. ATCC33591

| | | | | | | |
|---|---|---|---|---|---|---|
| −39.74% | 53.51% | −35.53% | −6.59% | −40.29% | −82.20% | −127.91% |
| −41.64% | 58.13% | −22.85% | 8.94% | −50.72% | −54.66% | −79.70% |
| −46.01% | 47.95% | −37.97% | −12.84% | 10.76% | −58.17% | −69.36% |
| −53.74% | 71.68% | −4.82% | −4.10% | −30.27% | −47.79% | −60.38% |
| −45.28% | 57.82% | −25.29% | −3.65% | −27.63% | −60.71% | −84.34% |

P. a ATCC27853

| | | | | | | |
|---|---|---|---|---|---|---|
| 84.47% | 72.46% | −65.13% | 20.89% | 65.93% | 5.25% | −38.69% |
| 21.94% | 68.72% | 23.35% | −163.74% | −61.13% | −50.98% | −15.25% |
| 70.40% | 42.01% | −38.45% | 50.40% | 43.30% | −10.37% | 2.97% |
| 66.33% | 78.09% | −43.58% | 27.90% | 10.55% | −24.63% | −10.04% |
| 60.79% | 65.32% | −30.95% | −16.13% | 14.67% | −20.18% | −15.25% |

E. coli

| | | | | | | |
|---|---|---|---|---|---|---|
| 61.81% | 71.55% | 62.88% | 22.74% | −8.94% | −164.16% | −78.99% |
| 50.23% | 68.19% | 60.15% | 50.00% | 57.15% | −78.08% | −17.36% |
| 54.03% | 58.06% | 65.55% | 46.45% | −2.29% | −59.41% | −45.90% |
| 47.10% | 51.80% | 53.96% | 50.42% | 14.28% | −89.21% | −72.50% |
| 53.29% | 62.40% | 60.64% | 42.40% | 15.05% | −97.71% | −53.69% |

### Experiment 2

S. a. ATCC33591

| | | | | | | |
|---|---|---|---|---|---|---|
| 59.54% | 72.96% | 53.71% | 15.33% | 8.38% | −53.92% | −88.09% |
| 56.34% | 62.52% | 21.92% | 12.93% | 19.15% | −14.20% | −59.52% |
| 52.78% | 65.93% | 37.01% | 24.38% | 33.03% | −20.80% | −71.15% |
| 53.24% | 53.51% | 33.51% | 16.55% | 20.44% | −23.67% | −48.41% |
| 55.48% | 63.73% | 36.54% | 17.30% | 20.25% | −28.15% | −66.79% |

P. a. ATCC27853

| | | | | | | |
|---|---|---|---|---|---|---|
| 2.57% | 68.34% | 5.24% | 27.88% | −14.59% | −75.24% | −41.39% |
| 38.24% | 74.92% | 20.99% | 24.22% | 10.76% | −0.32% | −23.42% |
| 35.44% | 68.48% | −4.60% | 23.21% | −59.29% | −29.17% | −52.90% |
| 53.85% | 40.58% | 8.23% | −186.87% | −179.55% | 0.51% | −3.32% |
| 32.52% | 63.08% | 7.47% | −27.89% | −60.67% | −26.06% | −30.25% |

E. coli

| | | | | | | |
|---|---|---|---|---|---|---|
| 73.70% | 74.52% | 79.99% | 42.88% | 47.41% | −145.36% | −146.16% |
| 71.32% | 81.67% | 72.38% | 58.29% | 46.13% | −17.77% | −39.49% |
| 45.21% | 73.00% | 68.24% | 55.60% | 58.20% | 25.12% | −39.89% |
| 55.55% | 73.78% | 77.73% | 72.05% | 52.11% | 24.09% | −4.80% |
| 61.45% | 75.74% | 74.59% | 57.21% | 50.96% | −28.48% | −57.59% |

Thus, the product Enzimed Prevent alone is consistently effective against E. coli and P. aeurginosa, but its effectiveness against S. aureus is variable. However, this Enzimed composition enriched with the DNAse of the present invention shows a routinely increased and constant activity, even against S. aureus. The other cleaning or even disinfecting compositions are less effective and sometimes even not effective at all on the biofilms tested.

1. A liquid composition for the prevention or removal of a biofilm, the liquid composition comprising one or more surfactant(s), one or more sequestering agent(s), and a phosphodiesterase having deoxyribonuclease (DNAse) activity, said phosphodiesterase being one or more DNAse(s) of HNH type and/or having at least 95% identity with a sequence selected from the group consisting of: SEQ ID NOs: 1-13.

2. The composition according to claim 1, wherein the DNAse has at least 95% identity with the sequence selected from the group consisting of: SEQ ID NOs: 1-13.

3. The composition according to claim 1, further comprising one or more enzymes selected from the group consisting of: a protease, a laccase, an amylase, a lipase, a cellulase, a mannanase, an enzyme having a β-1,6-N-acetylhexosaminidase (Dispersin B) activity, and any combination thereof.

4. The composition according to claim 1, wherein the one or more sequestering agent(s) is selected from the group consisting of: citrate, carboxymethylinulin, a phosphate, a phosphonate, tetrasodium glutamate diacetate (GLDA), trisodium methylglycine diacetate (MGDA), sodium iminodisuccinate (IDS), a gluconate, and any combination thereof.

5. The composition according to claim 1, further comprising glycerol and/or monopropylene glycol between 5 and 30% (weight:volume).

6. The composition according to claim 1, further comprising a preservative, an isothiazolinone, benzisothiazolinone, or a phenoxyethanol.

7. The composition according to claim 1, comprising at least 7% water (weight:volume).

8. The composition according to claim 1, comprising at least 60% water (weight:volume), wherein the composition further comprises β-1,6-N-acetylhexosaminidase (Dispersin B).

9. The composition according to claim 1, comprising at least 90% water (weight:volume) and a foaming surfactant.

10. A method for the removal of a biofilm comprising a lactic acid bacterium, wherein the lactic acid bacterium is:
selected from the group consisting of: Lacobacillus sp, Pediococcus sp, Lactococcus sp. Streptococcus sp, Teragenococcus sp, Leuconostoc sp, Oenococcus sp, and Bifidobacterium sp;
selected from the group consisting of: Listeria monocytogenes, Stenotrophomonas maltophilia, Bacillus cereus, and Bacillus subtilis; and/or
selected from the group consisting of: Pseudomonas fluorescens, Pseudomonas aeruginosa, Staphylococcus aureus, Escherichia coli, Klebsiella pneumoniae, Enterobacter cloacae, Citrobacter freundii, Staphylococcus epidermidis or Clostridium sp.

11. A liquid composition according to claim 1, wherein the biofilm is present on a tissue of a patient, wherein the tissue comprises the oral cavity, a mucous membrane, or a wound, or is in contact with an implant.

12. A method for the removal of a biofilm on a surface, the method comprising applying to said surface the liquid composition according to claim 1.

13. The method according to claim 12, wherein the surface is an internal surface and/or an external surface of an endoscope and the method further comprises applying a disinfectant to said surface.

14. The method according to claim 12, wherein the surface is that of a surgical instrument and the method further comprises applying a disinfectant to said surface and then applying a sterilizing heat treatment.

15. The method according to claim 13, wherein the disinfectant is peracetic acid.

16. A composition for the prevention or treatment of biofilms present on a substantially vertical surface, the composition comprising:
one or more enzymes selected from the group consisting of: a DNAse, a protease, a polysaccharidase, a lipase, a laccase, and any combination thereof; and
one or more surfactants, wherein at least one surfactant of the one or more surfactant is a foaming surfactant.

17. The composition according to claim 16, further comprising a surfactant having a quaternary amine, said surfactant having the quaternary amine being zwitterionic.

18. A method for the prevention or treatment of a biofilm on a substantially vertical surface, the method comprising the successive steps of:
applying the composition according to claim 16 to said substantially vertical surface for a predetermined time; and
adding a microbiocidal agent for a predetermined time.

19. The method according to claim 18, wherein the substantially vertical surface is an emergent part of a siphon.


SEQUENCE LISTING (18 sequences)

SEQ ID NO: 1 - protein, 182 aa, Bacillus sp.
LPPGTPSKSEAQSQLNALTVKPEDPMTGYSRDHFPHWISQGNGCNTRQIVLQRDADYYSGACPVTTGKWYSYFDGVIVYSPSEIDIDHIVPLAEAWRSGASSWTTEKRRSFANDLNGPQLIAVTASVNRSKGDQDPSTWQPPRAGARCAYAKWWINTKHRWGLHLQSSEKSSLQSMLNGCAY

SEQ ID NO: 2 - protein, 182 aa, Bacillus sp.
LPPGTPSKSEAQSQLNSLTVKTEDPMTGYSRDLFPHWSGQGSGCDTRQIVLQRDADYFTGTCPTTSGKWYSYFDGVIVYSPSEIDVDHIVPLAEAWRSGASSWTTEQRRAFANDLTGPQLIAVTASVNRSKGDQDPSTWQPPRAGARCAYAKWWINTKHRWNLHLQSSEKSSLQTMLNGCAY

SEQ ID NO: 3 - protein, 182 aa, Bacillus sp.
LPPGTPSKSEAQSQLNALTVKAEDPMTGYSRNLFPHWNSQGNGCNTRQLVLQRDADYYSGNCPVTSGRWYSYFDGVVVTSPSEIDIDHIVPLAEAWRSGASSWTTEKRKEFANDLNGPQLIAVTASVNRSKGDQDPSTWQPPRAAARCGYAKWWINTKYRWDLSLQSSEKSSLQTMLNTCSY

SEQ ID NO: 4 - protein, 182 aa, Bacillus sp.
LPPGTPSKSQAQSQLNALTVKAEDPMTGYSRNLFPHWSSQGNGCNTRQLVLQRDADYYSGNCPVTSGRWYSYFDGVVVTSPSEIDIDHIVPLAEAWRSGASSWTTEKRREFANDLNGPQLIAVTASVNRSKGDQDPSTWQPPRVAARCGYAKWWINTKYRWDLSLQSSEKSSLQTMLNTCSY

SEQ ID NO: 5 - protein, 182 aa, Bacillus sp.
FPPEIPSKSTAQSQLNSLTVKSEDAMTGYSRDKFPHWISQGDGCDTRQMVLKRDADYYSGSCPVTSGKWYSYYDGITVYSPSEIDIDHIVPLAEAWRSGASSWTTEKRRNFANDLNGPQLIAVTASVNRSKGDQDPSTWQPPRSGARCAYAKMWVNTKYRWGLHLQSAEKSGLESMLNTCSY

SEQ ID NO: 6 - protein, 182 aa, Bacillus sp.
FPPGTPSKSTAQSQLNSLTVKSEGSMTGYSRDKFPHWIGQGSGCDTRQLVLQRDADYYSGSCPVTSGKWYSYYDGVTFYDPSDLDIDHVVPLAEAWRSGASSWSTQKRKDFANDLSGPQLIAVSASSNRSKGDQDPSTWQPTRSGAACGYSKWWISTKHKWGLSLQSSEKNALQGMLNSCVY

SEQ ID NO: 7 - protein, 182 aa, Bacillus sp.
IPPGTPSKSAAQSQLDSLAVQSEGSMSGYSRDKFPHWIGQGNGCDTRQLVLQRDADYYSGDCPVTSGKWYSYFDGVQVYDPSYLDIDHMVPLAEAWRSGASSWSTQKREDFANDLDGPHLIAVTASSNRSKGDQDPSTWKPTRYSAHCGYAKWWINTKYVYDLNLQSSEKSALQSMLNTCSY

SEQ ID NO: 8 - protein, 182 aa, Paenibacillus mucilaginosus
LPPGTPSKSTAQSQLNSLTVKSESTMTGYSRDKFPHWTSQGGGCDTRQVVLKRDADYYSGSCPVTSGKWYSYYDGITVYSPSEIDIDHIVPLAEAWRSGASSWTTEKRQNFANDLGGPQLIAVTASSNRAKGDQDPSTWKPTRSGAHCAYAKWWINTKYRWGLHLQSSEKTALQSMLNTCSY

SEQ ID NO: 9 - protein, 182 aa, Bacillus sp.
TPPGTPSKSTAQTQLNALTVKTEGSMTGYSRDLFPHWISQGSGCDTRQVVLKRDADYYSGSCPVTSGKWYSYYDGVTFYDPSDLDIDHIVPLAEAWRSGASSWTTSKRQDFANDLSGPQLIAVSASTNRSKGDQDPSTWQPPRAGAACGYSKWWISTKYKWGLSLQSSEKTALQGMLNSCSY

SEQ ID NO: 10 - protein, 182 aa, Bacillus sp.
TPPVTPSKATSQSQLNGLTVKTEGAMTGYSRDKFPHWSSQGGGCDTRQVVLKRDADSYSGNCPVTSGSWYSYYDGVKFTNPSDLDIDHIVPLAEAWRSGASSWTTAQREAFANDLSGSQLIAVSASSNRSKGDQDPSTWQPPRAGAKCGYAKWWISTKSKWNLSLQSSEKTALQGMLNSCVY

SEQ ID NO: 11 - protein, 182 aa, Bacillus sp.
LPSGIPSKSTAQSQLNSLTVKSEGSMTGYSRDKFPHWISQGGGCDTRQVVLKRDADYYSGNCPVTSGKWYSYYDGISVYSPSEIDIDHVVPLAEAWRSGASSWTTTKRQNFANDLNGPQLIAVTASVNRSKGDQDPSTWQPPRYGARCAYAKMWINTKYRWDLNLQSSEKSSLQSMLDTCSY

SEQ ID NO: 12 - protein, 182 aa, synthetic construct
  VARIANT 1: note = L, F, I, T, P
  VARIANT 3: note = P, S
  VARIANT 4: note = G, E, V
  VARIANT 5: note = T, I
  VARIANT 9: note = S, A
  VARIANT 10: note = A, E, Q, T
  VARIANT 11: note = A, S
  VARIANT 13: note = S, T
  VARIANT 16: note = N, D
  VARIANT 17: note = A, S, G
  VARIANT 19: note = T, A
  VARIANT 21: note = K, Q
  VARIANT 22: note = A, P, T, S
  VARIANT 24: note = D, S, G
  VARIANT 25: note = P, T, A, S
  VARIANT 27: note = T, S
  VARIANT 32: note = D, N
  VARIANT 33: note = L, H, K
  VARIANT 38: note = N, S, I, T
  VARIANT 39: note = S, G
  VARIANT 42: note = S, G, N
  VARIANT 45: note = D, N
  VARIANT 49: note = I, L, V, M
  VARIANT 52: note = Q, K
  VARIANT 57: note = Y, S
  VARIANT 58: note = Y, F
  VARIANT 59: note = S, T
  VARIANT 61: note = N, A, T, S, D
  VARIANT 64: note = V, T
  VARIANT 66: note = S, T
  VARIANT 68: note = R, K, S
  VARIANT 73: note = F, Y
  VARIANT 76: note = I, V
  VARIANT 77: note = V, I, T, K
  VARIANT 78: note = V, F
  VARIANT 79: note = T, Y
  VARIANT 80: note = S, D, N
  VARIANT 83: note = E, D, N
  VARIANT 84: note = I, L
  VARIANT 86: note = I, V
  VARIANT 89: note = I, V
  VARIANT 104: note = S, T
  VARIANT 106: note = E, S, Q, A, T
  VARIANT 107: note = K, Q
  VARIANT 109: note = E, K, Q, R
  VARIANT 110: note = E, S, A, N, D
  VARIANT 116: note = N, T, G, S
  VARIANT 118: note = P, S
  VARIANT 124: note = S, T
  VARIANT 127: note = T, S, V
  VARIANT 130: note = S, A
  VARIANT 140: note = Q, K
  VARIANT 142: note = P, T
  VARIANT 144: note = A, V, S, Y
  VARIANT 145: note = A, G, S
  VARIANT 147: note = R, H, K, A
  VARIANT 149: note = G, A
  VARIANT 151: note = S, A
  VARIANT 153: note = W, M
  VARIANT 155: note = I, V
  VARIANT 156: note = N, S
  VARIANT 159: note = H, Y, S
  VARIANT 160: note = R, V, K
  VARIANT 161: note = W, Y
  VARIANT 162: note = D, G, N
  VARIANT 164: note = S, H, N
  VARIANT 168: note = S, A
  VARIANT 171: note = S, T, N
  VARIANT 172: note = S, A, G
  VARIANT 174: note = Q, E
  VARIANT 175: note = T, S, G
  VARIANT 178: note = N, D
  VARIANT 179: note = T, G, S
  VARIANT 181: note = S, A
XPXXXPSKXXXQXQLXXLXVXXEXXMXGYSRXXFPHWXXQGXGCXTRQXVLXRDADXXSGXCPXTXGXWYSYXDGXXXXXPSXXDXDHXVPLAEAWRSGASSWXTXXRXXFANDLXGXQLIAVXASXNRXKGDQDPSTWXPXRXXAXCXYXKXWXXTKXXXXLXLQSXEKXXLXXMLXXCXY

SEQ ID NO: 13 - protein, 182 aa, synthetic construct
  VARIANT 1: note = I
  VARIANT 10: note = E, A, Q, T
  VARIANT 19: note = T, A; note = T,A
  VARIANT 21: note = K, Q
  VARIANT 22: note = A, P, T, S
  VARIANT 24: note = D, S, G
  VARIANT 57: note = Y, S
  VARIANT 58: note = Y, F
  VARIANT 61: note = N, A, T, S, D
  VARIANT 76: note = I, V
  VARIANT 77: note = V, I, T, K
  VARIANT 124: note = S, T
  VARIANT 127: note = T, S, V
  VARIANT 130: note = S, A
  VARIANT 168: note = S, A
  VARIANT 171: note = S, T, N
  VARIANT 172: note = S, A, G
  VARIANT 174: note = Q, E
  VARIANT 175: note = T, S, G
  VARIANT 178: note = T, G, S
  VARIANT 179: note = T, G, S
  VARIANT 10: note = E, Q
  VARIANT 32: note = D, N
  VARIANT 33: note = L, H, K
  VARIANT 109: note = Q, R
  VARIANT 110: note = N, D
LPPGTPSKSXAQSQLNALXVXXEXSMTGYSRXXFPHWSSQGGGCDTRQVVLKRDADXXSGXCPVTSGKWYSYFDGXXFTNPSDLDIDHIVPLAEAWRSGASSWTTSKRXXFANDLNGPQLIAVXASXNRXKGDQDPSTWQPPRAAARCAYSKWWISTKYKWGLSLQSXEKXXLXXMLXXCAY

SEQ ID NO: 14 - protein, 182 aa, unidentified
LPPGTPSKSAAQSQLNALTVKTEGSMSGYSRDLFPHWISQGSGCDTRQVVLKRDADSYSGNCPVTSGSWYSYYDGVTFTNPSDLDIDHIVPLAEAWRSGASSWTTSKRQDFANDLSGPQLIAVSASTNRSKGDQDPSTWQPPRSGAACGYSKWWISTKYKWGLSLQSSEKTALQGMLNSCSY

SEQ ID NO: 15 - protein, 182 aa, unidentified [sequence omitted]

SEQ ID NO: 16 - protein, 182 aa, unidentified; identical to SEQ ID NO: 9

SEQ ID NO: 17 - protein, 182 aa, unidentified; identical to SEQ ID NO: 9

SEQ ID NO: 18 - protein, 182 aa, unidentified; identical to SEQ ID NO: 1